(19) United States
(12) Patent Application Publication
(10) Pub. No.: US 2004/0194351 A1
(43) Pub. Date: Oct. 7, 2004

(54) Footwear

(76) Inventor: Alvaro Z. Gallegos, Albuquerque, NM (US)

Correspondence Address: THE MATTHEWS FIRM, 2000 BERING DRIVE, SUITE 700, HOUSTON, TX, 77057, US

(21) Appl. No.: 10/409,751

(22) Filed: Apr. 7, 2003

(51) Int. Cl. A43B 13/38; A43B 3/24; A43B 23/00; A61F 5/14

(52) U.S. Cl. 36/140; 36/148; 36/44

(57) ABSTRACT

The present invention relates to footwear which has improved shock absorbency due to a cushioning and has superior support due to a rigid or semi-rigid orthotic plate. The orthotic plate may be removably or securably attachable to the upper, and the padding may be removably or securably attachable to the orthotic plate. The cushioning may further may have supporting blades incorporated therein to counteract any undesired deforming characteristics of the cushioning and/or a bottom plate may be securably or removably attachable to the bottom side of the cushioning. The bottom plate may also have gripping elements which are permanently or removably attachable to the plate to compensate for wear, or for a change in activities. Further, the upper may be removably and attachable and interchanged with other types of removably attachable uppers.

### TECHNICAL FIELD

The present invention relates to footwear which has improved shock absorbency, comfort, and versatility.

### PRIOR ART

The following patents are believed to be prior art of the current invention: U.S. Pat. No. 4,377,042; U.S. Pat. No. 5,317,822; U.S. Pat. No. 5,353,522; U.S. Pat. No. 5,410,821; U.S. Pat. No. 5,615,497; U.S. Pat. Nos. 5,996,252 and 5,727,334; and U.S. Pat. No. 6,345,454.

### BRIEF DESCRIPTION OF THE DRAWINGS

FIG. 1 is a side view of an embodiment of the footwear;

FIG. 2 is a bottom view of an embodiment of the footwear;

FIG. 3 is a side view of an embodiment of the footwear;

FIG. 4 is a perspective view of an embodiment of the footwear;

FIG. 5 is a side view of a footwear embodiment showing a multipiece orthotic plate;

FIG. 6 is a side view of a footwear embodiment with a plate and gripping members;

FIG. 7 is a view of an embodiment of high-top footwear with a plate and gripping members;

FIG. 8 is an expanded view and an embodiment of footwear;

FIG. 9 is an embodiment of a ski boot;

FIG. 10 is the bottom view of an embodiment of the footwear; and

FIGS. 11a-c is a side view of an embodiment of the gripping members of an embodiment of the footwear.

### DETAILED DESCRIPTION OF THE PREFERRED EMBODIMENT

For purposes of the description of this invention, the terms “upper,” “lower,” “right,” “left,” “vertical,” “horizontal,” “top,” “bottom,” and other related terms shall be defined in relation to embodiments of the present invention as it is shown and illustrated in the accompanying figures. However, it is to be understood that the invention may assume various alternative structures and processes and still be within the scope and meaning of this disclosure. Further, it is to be understood that any specific dimensions and/or physical characteristics related to the embodiments disclosed herein are capable of modification and alteration while still remaining within the scope of the present invention and are, therefore, not intended to be limiting.

One way to increase the comfort and also shock absorbency of footwear is to increase the amount of shock absorbing material and/or by using padding comprised of various materials, especially foam. This is notably advantageous in athletic footwear worn by runners or other athletes who are involved in rigorous sporting activities since the wearer's footwear and body encounters substantial forces especially from the hard surfaces which are contacted by the wearer's footwear. And, even in the footwear of this invention worn by non-athletes, padding makes the footwear more comfortable.

In conjunction with the superior shock absorbing advantages of the padding, the padding may be removably attached and interchanged with padding types and/or varying thicknesses that are used for special purposes such as marathons or long distance runs. Also, when the padding shows wear or loses some of the shock absorbing capabilities or has changed compressibility, the worn padding can be replaced with new padding. By also making parts or components of the footwear replaceable, the durability of the footwear may also be enhanced.

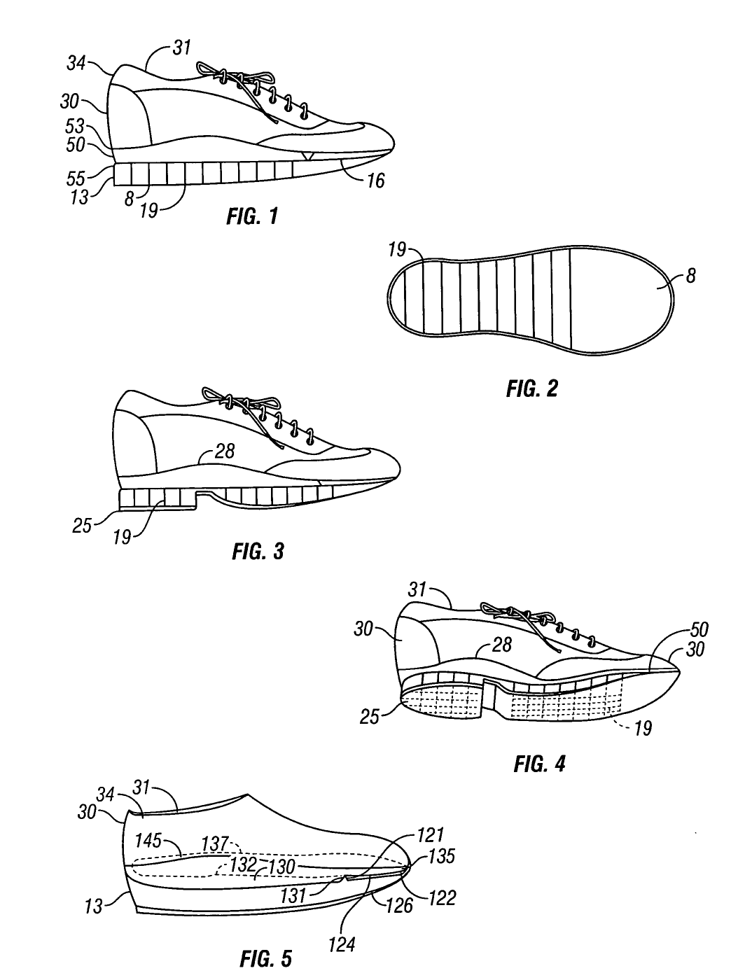

Several variations of footwear of this invention are illustrated in FIGS. 1-11. FIG. 1 shows one variation of the footwear with an upper 30 having an inside 31 and an outside 34. The footwear also has an orthotic plate 50 that has a first surface 53 and a second surface 55 and a layer of padding 13, having a top side 16 and a bottom side 8 that in an embodiment also serves as the wear surface. Also, in an embodiment, there are stabilizing blades 19 within the padding. The style of the upper 30 and the manner of securing it to orthotic plate 50 will vary depending on the design of the footwear. The upper 30 includes all types of footwear, and the invention is especially advantageous in closed footwear such as an athletic shoes, work boots, hiking boots, etc. The upper 30 may be construction of any type of material such as canvas, leather, synthetic leather, vinyl, plastic, etc., or a combination thereof, and any other materials known to or used by one of ordinary skill in the art. The upper 30 can be secured to the orthotic plate using conventional techniques such as gluing, but it could also be stitched and/or secured by adhesive. Alternatively, the upper 30 may be removably attached to the orthotic plate such as by a stud and aperture connection, Velcro®, i.e., mated hooks and loops, as shown in FIG. 8. As an alternative, other means of attachment such as fasteners, adhesives, magnets, a wedge and aperture connection, a screw, a bolt, a hinge, a staple, a nut, studs, snaps, wedges and a combination thereof may be used, to allow replacement or interchanging of the orthotic plate. An insole 145 which may be made of textile material such as cotton, felt, linen, polyester, or leather, silicon, or other such materials that one of the ordinary skill of the art uses as insoles may also be placed inside the footwear as in, e.g., FIG. 5.

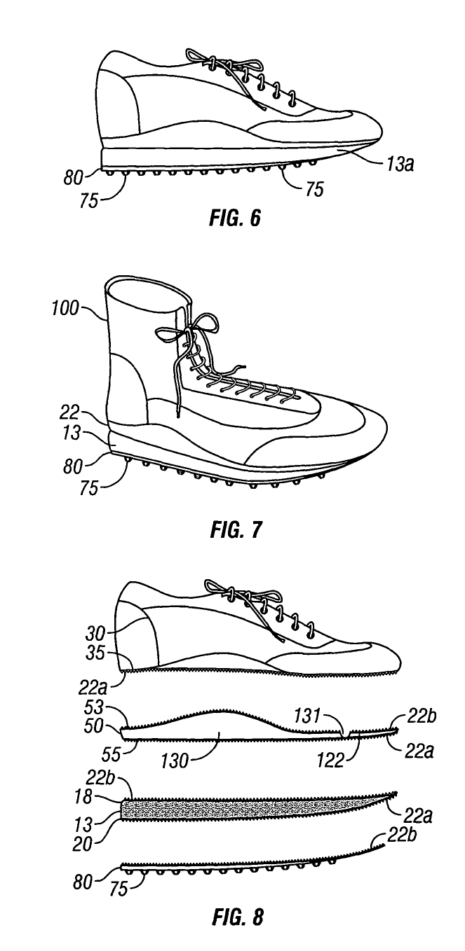

The orthotic plates of the footwear provide superior support, and typically have a raised arch area 28, see, e.g., FIGS. 3-4, and may also have a flexible or a semi-flexible front portion. Further, the orthotic plate has a first surface 124 and second surface 126. The orthotic plate may be a unitary piece or may be formed of more than one piece, with for example, a first piece 122 that typically extends to or beyond the front toe area 135, and a second piece 130 that extends to the rear most portion of the footwear, and the second piece 130 which is preferably rigid or nearly rigid forms a hinge-like area 131 adjacent to the end 121 of the first piece 122 that is preferably semi-flexible or flexible. E.g., FIG. 5. The orthotic plate typically extends from a metatarsal area 137 of a wearer's foot, to the rearmost portion of the footwear generally proximate to the heel area. In other embodiments, a bottom plate 80 may also be incorporated below the padding, forming a two-plate system. See FIGS. 6-8.

The footwear of this invention has cushioning 13 or padding below the orthotic plate 50. There may also be an undersole or outsole 25, see FIGS. 3-4, beneath the cushioning or padding 13, but the padding may also serve as the wear surface 8. See FIGS. 1-2. The undersole or outsole 25 may be fixedly or removably attached to the padding by the various ways known to one skilled in the art. The padding 13 can be removable, changeable, and replaceable and is preferably provided in a shape substantially similar and/or identical to the shape of the orthotic plate, such that it will cover the lower surface of the entire orthotic plate. In other embodiments, the padding can extend at least the length and the width of the footwear, although in some embodiments, the padding may be thinner and/or tapered 13a in the toe area of the footwear. See, e.g., FIG. 6. The padding may be of a variety of heights and may vary depending upon the wearer's needs and/or uses as well as the type of padding utilized. Preferably, the padding is between about ½ inch to about 3½ inches in height in its uncompressed state, but may be of a lesser or greater height as well. In the embodiments without a bottom plate, e.g., FIGS. 1-4, the padding is preferably about one ½ inch to about 3½ inches in height and in the embodiments with a bottom plate 80, see, e.g., FIGS. 6-8, the padding is preferably about ½ inch to about 2 inches in height, but may be of other heights as well.

The padding or cushioning layer 13 may comprise a variety of materials especially foamed materials which have elastic or rebounding properties, such as materials comprised of silicon, neoprene, natural rubber foams, synthetic rubber foams and polyurethane, polyether and polyester foams neoprene, Vinyl Nitrile, Styrene-Butadiene Rubber (SBR), Polyethylene (PE), ethyl vinyl acetate (EVA), ethylene propylene terpolymer (EPT), EPT/PE/Butyl Rubber, Neoprene/EPT/SBR, epichlorohydrin (ECH), and nitrile (NBR) or a combination thereof, or other cushioning materials known or used by one skilled in the art. Alternatively, in the two plate system other means can be used as cushioning such as leaf and coil wound springs, with or without padding. (not shown) The density and cell characteristics of the padding of the foam material are believed to be critical features in terms of providing the appropriate cushioning and rebound characteristics for cushioning and may vary depending upon the type of activity of footwear in question.

Preferably the cushioning or padding, such as foam, has a low to medium density so it is deformable. A low density padding comprises material within the range of about 0.08 g/cm³ to about 0.50 g/cm³. An even more preferred range of densities for padding is material between about 0.1 g/cm³ to 0.30 g/cm³.

The padding 13 may be constructed of a closed-cell foam material, having a density in the range of about 0.08 g/cm³ to 0.50 g/cm³, or of other suitable densities known to one-skilled in the art. Alternatively, open closed-cell foam material, having a density in the range of about 0.08 g/cm³ to 0.40 g/cm³ may be used, or of other suitable densities known to one skilled in the art.

Closed-cell foam material, in general, may demonstrate a greater resistance to wear as compared to open-cell foams. Open cell foam is a material where the open-air chambers in the foam are interconnected. This makes for extremely soft and highly compressible foam. While open-cell foam may be very comfortable, it has some disadvantages because of the high compressibility of the foam, especially if it is of a density which is too low or of a material that absorbs water. In contrast, the open air chambers in the closed-cell foam are completely surrounded by foam and not interconnected. As a result, the closed cell foam does not compress easily, yet provides good shock absorbency since the air pockets in the foam are completely encapsulated.

As a further alternative, since open-cell foam is typically more comfortable, i.e., more compressible and since closed-cell foam provides firmer support, a dual or multi-density padding may also be used, alternating layers of closed-cell foam and open-cell foam, with the closed cell foam giving stability and the open cell foam giving a cushioning effect. Depending upon the materials used and the characteristic desired, the alternating layers may each be of about 0.25 inches to about 1 inch in thickness, but may also be of other thicknesses.

The foam padding may be fabricated by pouring foamed materials into a frame that is heated and processed into sheets that are then cut to size. However, the cost of cutting and shaping foam is high and results in significant material wastage. To avoid wasting materials, the padding is preferably molded for each size and style of footwear. Additionally, a portion of the foam may be compression molded.

In some cases, depending upon the characteristics of the foam materials and the height of foam used, the materials may have undesired deformation. To counteract any undesired deformation and/or to control the characteristics of the low density foam, the padding may further may have supporting or stabilizing blades incorporated therein. See FIGS. 1-4. In these circumstances, the stabilizing blades 19, may be incorporated into the foam and/or a semirigid plate or rigid plate may be positioned below the underside of the cushioning with or without stabilizing blades in the cushioning.

The stabilizing blades 19 are made of rigid, semi-rigid flexible, and/or semi-flexible materials such as rubber, plastic or denser foams made from EVA, silicon, neoprene, and the like incorporated into the foam, as well as plastic, rubber, metal, metal alloys, vinyl. There are a multitude of ways the stabilizing blades 19 may be arranged or position within the foam and/or cushioning. For example, FIGS. 1-2 shows footwear wherein the stabilizing blades are parallel to one another. FIGS. 3-4 in contrast, show footwear wherein the stabilizing blades are arranged in a cross hatch or grid fashion. The blades may be placed throughout the foam or may be concentrated in certain areas, or alternatively spaced consistently, or fairly consistently. The blades 19 are preferably about ½ inch to about ½ inches apart, but may be of other distances. The blades may also be straight, waved, and/or curved. The blades could also be arranged in a variety of other ways by example, but without limitation such as triangles, rectangles, hexagons, octagons (not shown). The blades may also zigzag through the foam, and may alternatively be arranged in a wave like patterns (not shown). There are many ways the stabilizing blades may be incorporated into the foam. They may be incorporated into the foam at the time of the original manufacture of the foam, or heat bonded or glued to already made foam. There are other means which also may be used to stabilize the foam such as a plurality of small springs.

As an alternative or in addition to the stabilizing blades, a rigid or semirigid bottom 80 plate may be placed beneath the foam layer. See FIGS. 6-8. If a bottom plate is used along with the orthotic plate, the footwear of this invention has a two-plate system. The bottom plate may comprise metal, plastic, compressed EVA or other compressed foams, silicon, or neoprene foam. The bottom plate may serve as the wear surface, or may alternatively have an outsole further attached to the outer surface of the plate (not shown). The plate may be glued or heat bonded to the foam, may be removably attached through means such as a plurality of hooks and eyes such as Velcro® or may alternatively have telescoping pins with an enlarged end like that of a nail or screw head that attaches at one end to the orthotic plate and attaches at the other end to the plate. The bottom plate 80 may also have gripping members 75 such as spikes, cleats, studs, and the like permanently or removably attachable to the bottom plate 80.

FIG. 8 shows footwear that is disassembled and shows the first surface 53 of the orthotic plate 50 separated from the bottom surface 35 of the upper 30, the padding 13 separated from the second surface 55 of the orthotic plate 50, and the bottom plate 80 separated from the cushioning 13. In use, these counterparts are mated and fastened by applying pressure to the hooks and loops, or other fastening means which engage each other. The orthotic plate 50, which as shown in FIG. 8 comprises a first piece 122, a second piece 130 and the hinged area in between 131. The foam padding 13 may also be removably attached to the orthotic plate such as by a stud and aperture connection, Velcro®, i.e., mated hooks and loops, fasteners, adhesives, magnets, a wedge and aperture connection, a screw, a bolt, a hinge, a staple, a nut, studs, snaps, wedges and a combination thereof or by other means known to one skilled in the art. If a bottom plate 80 is also used, it may also be removably attachable to the padding by the same foregoing means or means such as Velcro® 22 or other hook-and-loop type fasteners. Additionally, the orthotic plate 50, may also be removably attachable to the bottom 35 of the upper 30. Furthermore, the upper may be replaced with another type or style of upper, such as an upper that is a sandal or open toed shoe, a dress upper, a boot, and other types of uppers known to one skilled in the art that have been modified to be removably attachable such as by the means described herein for the orthotic plate and cushioning.

As shown FIG. 8, the top 18 and the bottom 20 of the padding 13 has a layer, strip, or portion 22 of one of the counterpart elements 22a, 22b of the hook-and-loop type fasteners, such as Velcro®. Above the top of the cushioning, the orthotic plate also has at least one of the counterpart elements 22b of the hook-and-loop type fasteners, such as Velcro®, which is compatible with that the counterpart elements 22a on the bottom surface 35 of the upper 30. Alternatively, any of the components that are covered with Velcro®, could also have portions of both the hook and loop type elements as long as there are corresponding counterparts on the surface of the adjacent component which is removably attachable. If Velcro® or Velcro®-like material is used, it is preferably attached to the top or bottom of the components such as the orthotic plate, front portion 122, padding 13, or bottom plate 80 by adhesive, glue, or other suitable means, such as stitching and heat bonding. The entire surface of the orthotic plate and cushioning or other such components may be covered by Velcro® or other attaching means or alternatively only portions such as the perimeter of the components may have Velcro® or attachment portions. It is preferred that the hook-and-loop fasteners used in the present invention be selected from the strongest of those available on the market to resist separation while worn. Still, even when using such strong fasteners the wearer can remove worn padding and quickly install replacement padding or components by separating and then pulling the padding from the bottom of the footwear and aligning and pressing the replacement padding in place. The pressure applied by the wearer's weight further anchors and/or secures the padding and other removable attachable components to the footwear. Once all the layers and/or counterparts which are removably attachable, interchangeable, and replaceable are connected together with the upper, they look like and function like conventional footwear.

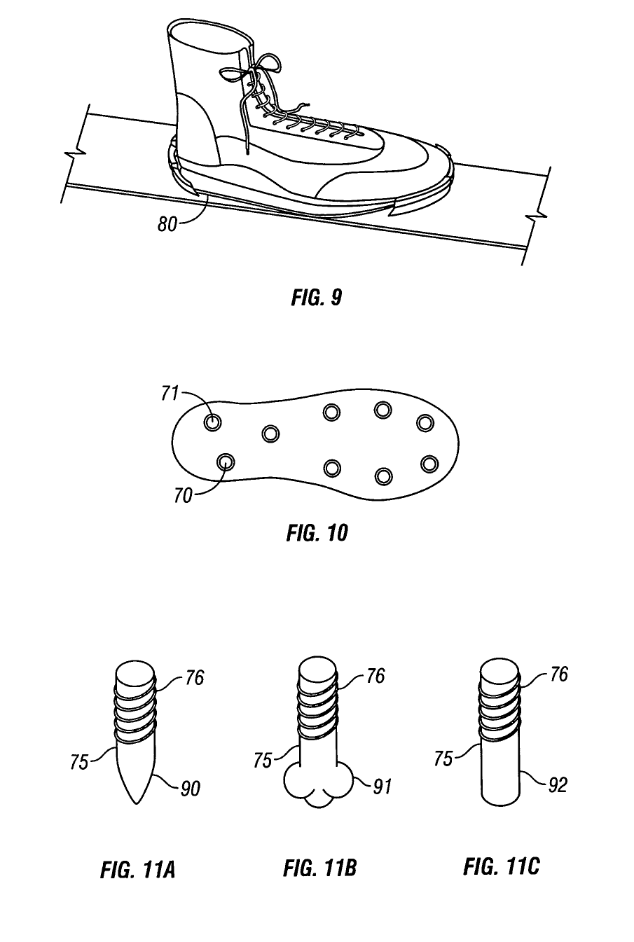

The bottom plate 80 may also be permanently or removably attached to the padding 13 such as by a stud and aperture connection, Velcro®, i.e., mated hooks and loops, fasteners, adhesives, magnets, a wedge and aperture connection, a screw, a bolt, a hinge, a staple, a nut, studs, snaps, wedges and a combination thereof or by other means known to one skilled in the art. In the embodiment having a plate below the cushioning, the plate preferably extends substantially at least along the length of the cushioning, but may also be tapered upwardly if the foam padding is also tapered or the toe area. Additionally, in some cases, the plate is the wear surface of the footwear. See FIG. 9.

Again, the bottom plate 80 may also have gripping members 75 such as spikes, cleats, studs or other such gripping members which are permanent or removably attachable to the bottom plate 80 that may be changed for different uses such as golf, soccer, rugby, football and other sports or activities where such footwear is helpful or useful, or removed altogether for other activities where gripping members are unnecessary or unwanted. See FIGS. 6-8, 10. The gripping members 75 are preferably removably attached to the bottom plate 80 shoe by inserting a threaded end 76 of the gripping member 75 into an aperture 70 in the bottom plate that has corresponding threading 71 and then turning or screwing in the gripping member 75 until it is inserted. See FIGS. 8, 10. Alternatively, the gripping members 75 may be snapped into the apertures and then removed such as by a tool. (not shown) The gripping members may comprise a variety of shapes such as a spike end 90, see FIG. 11a, multi-lobed end 91, see FIG. 11b, a rounded end 92, see FIG. 11c, or other shapes known or used by one skilled in the art. FIGS. 7 and 9 show embodiments of footwear that have a high top 100, and FIG. 9 has a two-plate system, but no gripping members, although the bottom plate may have a textured surface (not shown). Of course footwear with gripping members may have a high top.

In this invented footwear, the wearer will experience increased comfort and reduced stress when wearing footwear with gripping members, especially when the footwear is worn for several hours and/or when the wearer is involved in rigorous activities and/or when the ground surface is hard. In fact, the gripping members may be hardly noticeable to the wearer, unlike those in conventional footwear.

This invention also contemplates a method of making footwear comprising: providing an upper, obtaining an orthotic plate having a first surface and a second surface, wherein the first surface is removably or securably attachable to the upper, and adding at least one layer of cushioning having a top side and a bottom side, wherein the top side is removably or securably attachable to the second surface of the orthotic plate.

This method may also further comprise the step of attaching an orthotic plate to the upper, and attaching the cushioning to the orthotic plate. Additionally, in this method, a bottom plate may be obtained, wherein the plate has an upper side and a lower side, wherein the upper side is removably or securably attachable to the bottom side of the cushioning, and the plate is attached to the cushioning. Also, this method may further comprise the steps of providing at least one aperture in the bottom plate, and inserting a gripping member into at least one aperture. Also, this method may further comprise the step of removing the gripping member from the aperture.

The method may also further comprise separating at least the removably attachable padding and/or orthotic plate from the upper, and replacing the removed padding and/or orthotic plate with other removably attachable padding and/or another orthotic plate.

Additionally, the method may also comprise the steps of separating at least the upper from the orthotic plate, exchanging the separated upper with another upper, and attaching another upper to the orthotic plate.

The above description is that of preferred embodiments of the invention. Various alterations and changes can be made without departing from the spirit and broader aspects of the invention as defined in the appended claims, which are to be interpreted in accordance with the principles of patent law including the doctrine of equivalents.

What is claimed is:

1. Footwear comprising:
an upper;
an orthotic plate with a first surface and a second surface, wherein said first surface is removably or securably attachable to said upper; and
padding having a top side and a bottom side, wherein said top side is removably or securably attachable to said second surface of said orthotic plate, and wherein said padding is about ½ inch to 3½ inches in height.

2. The footwear of claim 1, having padding selected from the group comprised of silicon; neoprene; rubber; sponge; polyurethane; polyether; polyester; vinyl nitrile; ethyl vinyl acetate (EVA); ethylene propylene terpolymer (EPT); EPT/PE/Butyl Rubber; Neoprene/EPT/SBR; epichlorohydrin (ECH); and nitrile (NBR); or a combination thereof.

3. The footwear of claim 2, wherein said padding comprises foam.

4. The footwear of claim 3, having foam padding of about ½ inch to about 2 inches in height.

5. The footwear of claim 3, having foam padding of about 2 inches to about 3½ inches in height.

6. The footwear of claim 3, having open cell foam padding.

7. The footwear of claim 3, having closed cell foam padding.

8. The footwear of claim 3, having at least one layer of open cell foam padding and at least one layer of closed cell foam padding.

9. The footwear of claim 2, having padding constructed of foam with a density of about 0.08 g/cm³ to 0.40 g/cm³.

10. The footwear of claim 1, having an orthotic plate which is a unitary piece or which comprises at least two pieces.

11. The footwear of claim 3, wherein said orthotic plate is a rigid or semi-rigid piece extending at least from a metatarsal area of a wearer's foot to a rear area of the foot, the orthotic plate extending at least approximately two-thirds of a total horizontal length of the foot as measured from a toe area of the foot to the rear area of the foot.

12. The footwear of claim 2, wherein said orthotic plate and/or padding is removably and/or replaceably attached by hook and loop type fasteners.

13. Footwear comprising:
an upper;
an orthotic plate with a first surface and a second surface, wherein said first surface is removably or securably attachable to said upper;
a layer of cushioning having a top side and a bottom side wherein said top side is removably or securably attachable to said second surface of said orthotic plate;
a plurality of stabilizing blades within said cushioning; and
wherein said cushioning is about 1 inch to about 3 inches in height.

14. The footwear of claim 13, wherein said blades are made of material selected from the following group comprised of plastic; rubber; compressed foam; metal; metal alloy; vinyl and a combination thereof.

15. The footwear of claim 13, wherein said orthotic plate is a rigid or semi-rigid piece extending at least from a metatarsal area of a wearer's foot to a rear area of the foot, said orthotic plate extending at least approximately two-thirds of a total horizontal length of the foot as measured from a toe area of the foot to the rear area of the foot.

16. The footwear of claim 14, wherein said blades are rigid or semirigid.

17. The footwear of claim 14, wherein said blades are flexible or semiflexible.

18. The footwear of claim 14, wherein said blades are parallel to one another and/or wherein at least a portion of said blades are perpendicular to one another.

19. The footwear of claim 14, having cushioning of material selected from the group comprised of silicon; neoprene; rubber; sponge; polyurethane; polyether; polyester; vinyl nitrile; ethyl vinyl acetate (EVA); ethylene propylene terpolymer (EPT); EPT/PE/Butyl Rubber; Neoprene/EPT/SBR; epichlorohydrin (ECH); and nitrile (NBR); or a combination thereof.

20. The footwear of claim 14, wherein said blades are straight.

21. The footwear of claim 14, wherein said blades are curved and/or wavy.

22. Footwear comprising:
an upper;
an orthotic plate having a first surface and a second surface, wherein said first surface is removably or securably attachable to said upper,
at least one layer of cushioning having a top side and a bottom side, wherein said top side is removably or securably attachable to said second surface of said orthotic plate; and wherein said cushioning is about ½ inch to about 2½ inches in height; and
a bottom plate having a upper side and a lower side, wherein said upper side is removably or securably attachable to said bottom side of said cushioning.

23. The footwear of claim 22, having a first attaching element disposed beneath said upper and a second attaching element disposed on the first surface of said orthotic plate, wherein said upper capable of being detached from said orthotic plate and/or reattached to said orthotic plate.

24. The footwear of claim 23, wherein said upper is capable of attaching or reattaching to said orthotic plate by pressing said upper into said first surface of said orthotic plate after said upper and said orthotic plate are aligned.

25. The footwear of claim 23, having a first attachment means disposed below said second surface of said orthotic plate and a second attachment means on the top side of said padding, said orthotic plate capable of being detached from said padding and/or capable of being reattached to said padding.

26. The footwear of claim 23, wherein said upper side of said bottom plate is removably attachable to said cushioning.

27. The footwear of claim 23, wherein said orthotic plate is a rigid or semi-rigid piece extending at least from a metatarsal area of a wearer's foot to a rear area of the foot, said orthotic plate extending at least approximately two-thirds of a total horizontal length of the foot as measured from a toe area of the foot to the rear area of the foot.

28. The footwear of claim 23, wherein said cushioning is comprised of material from the group comprised of silicon; neoprene; rubber; sponge; polyurethane; polyether; polyester; vinyl nitrile; ethyl vinyl acetate (EVA); ethylene propylene terpolymer (EPT); EPT/PE/Butyl Rubber; Neoprene/EPT/SBR; epichlorohydrin (ECH); and nitrile (NBR); or a combination thereof.

29. The footwear of claim 22, wherein said cushioning is capable of attaching and/or reattaching to said footwear by pressing said cushioning into said second surface of said orthotic plate.

30. The footwear of claim 22, having an upper attaching element disposed on the upper side of said bottom plate and a lower second attachment means on the bottom side of said cushioning, said first and said second fastening elements being constructed so that said bottom plate can be detached from and/or reattached to said padding.

31. The footwear of claim 22, wherein said bottom plate has a plurality of apertures for receiving gripping members, and wherein said gripping members are removably attachable to said bottom plate.

32. The footwear of claim 22, wherein said bottom plate has a plurality of gripping members and wherein said gripping members are integral with said bottom plate.

33. The footwear of claim 28, wherein said cushioning comprises foam padding about ½ inch to about 2½ inches in height.

34. The footwear of claim 33, having open cell and/or closed cell foam padding.

35. The footwear of claim 34, having supporting blades with said foam padding.

36. The footwear of claim 28, wherein said cushioning comprises at least one layer of open cell foam and at least one layer of closed cell foam.

37. The footwear of claim 22, wherein said cushioning comprises at least one spring between said orthotic plate and said bottom plate.

38. The footwear of claim 37, further comprising foam padding between and/or integral with said at least one spring.

39. A method of making footwear comprising:
providing an upper;
obtaining an orthotic plate having a first surface and a second surface, wherein said first surface is removably or securably attachable to said upper; and
adding at least one layer of cushioning having a top side and a bottom side, wherein said top side is removably or securably attachable to said second surface of said orthotic plate.

40. The method of claim 39, further comprising the steps of:
attaching said orthotic plate to said first surface of said upper; and
attaching said cushioning to said second surface of said orthotic plate.

41. The method of claim 39, further comprising the steps of:
obtaining a bottom plate having an upper side and a lower side, wherein said upper side is removably or securably attachable to said bottom side of said cushioning; and
attaching said plate to said cushioning.

42. The method of claim 39, further comprising the steps of:
providing at least one aperture in said lower side of said bottom plate; and inserting a gripping member into said at least one aperture.

43. The method of claim 39, further comprising the steps of:
separating at least said removably attachable padding and/or orthotic plate from said upper; and
replacing said removed padding and/or orthotic plate with other removably attachable padding and/or another orthotic plate.

44. The method of claim 40, further comprising the steps of:
separating at least said upper from said orthotic plate;
exchanging said separated upper with another upper; and
attaching said another upper to said orthotic plate.

45. The method of claim 42, further comprising the steps of:
providing a plurality of apertures in said lower side of said bottom plate; and
inserting at least one gripping member into each said aperture.

46. The method of claim 42, further comprising the step of:
removing said gripping member from said at least one aperture and/or removing all said gripping members from said apertures.
